(19) United States
(12) Patent Application Publication
(10) Pub. No.: US 2022/0088626 A1
(43) Pub. Date: Mar. 24, 2022

(54) ELECTOSTATIC SPRAY WAND

(71) Applicant: Minuteman International, Inc., Pingree Grove, IL (US)

(72) Inventors: Jeremy C. Stoddard, Holland, MI (US); Brent A. Caudill, Holland, MI (US); John M. Hill, Naperville, IL (US)

(73) Assignee: Minuteman International, Inc.

(21) Appl. No.: 17/025,070

(22) Filed: Sep. 18, 2020

(51) Int. Cl. B05B 5/053; B05B 5/16; B05B 12/00; A61L 2/18; A61L 2/26

(52) U.S. Cl. CPC B05B 5/0533; B05B 5/0531; B05B 5/1691; A61L 2202/16; A61L 2/18; A61L 2/26; A61L 2202/15; B05B 12/002

(57) ABSTRACT

An electrostatic spraying wand includes a housing and a power source for operating an electrostatic generator. The electrostatic generator supplies a substantially high voltage to an electrode where a fluid is supplied to the electrode though a solution supply line. Thereafter, the charged solution is directed through a nozzle assembly. Since the electrostatic spraying wand supplies a charge to the cleaning fluid within the wand it can be used with a variety of devices enabling the charged solution to kill pathogens and other germs.

### FIELD OF THE INVENTION

The present invention relates generally to a spraying device and more particularly to an electrostatic sprayer for charging cleaning fluid before leaving a spraying wand.

### BACKGROUND

Electrostatic spray surface cleaning is the process of spraying an electrostatically charged mist onto surfaces and objects. Electrostatic spray uses a specialized solution that is combined with air and atomized by an electrode inside the pumping device. Subsequently, the spray contains positively charged particles that are able to aggressively adhere to surfaces and objects. Because the particles in the spray are positively charged, they cling to and coat any surface they are aimed at.

For awkwardly shaped objects or hard to reach places, cleaning staff only have to point and spray. The nature of the mist allows it to coat surfaces evenly, and envelope objects—even if the mist is only sprayed from one side. After the spray is applied, the sanitizing agent works to disinfect the covered surfaces. For this reason, electrostatic spray is an excellent solution for germ, pathogen and contaminant ridden areas.

One problem associated with electrostatic devices is the charging device is integrated into the cleaning device. Accordingly, there are no sprayers available that can be used with spraying devices that have no means by which to charge the cleaning spray. Accordingly, new solutions are required to meet this demand.

### BRIEF DESCRIPTION OF THE FIGURES

The accompanying figures, where like reference numerals refer to identical or functionally similar elements throughout the separate views and which together with the detailed description below are incorporated in and form part of the specification, serve to further illustrate various embodiments and to explain various principles and advantages all in accordance with the present invention.

FIG. 1 is a perspective view showing a spraying device with wand.

FIG. 2A is an electrostatic spraying wand in accordance with an embodiment of the invention.

FIG. 2B is a cross-sectional view of the electrostatic spraying wand as shown in FIG. 2A.

FIG. 2C is an exploded view of the electrostatic spraying wand in accordance with FIG. 2A.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

### DETAILED DESCRIPTION

Before describing in detail embodiments that are in accordance with the present invention, it should be observed that the embodiments reside primarily in combinations of method steps and apparatus components related to an electrostatic spray wand. Accordingly, the apparatus components and method steps have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

In this document, relational terms such as first and second, top and bottom, and the like may be used solely to distinguish one entity or action from another entity or action without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

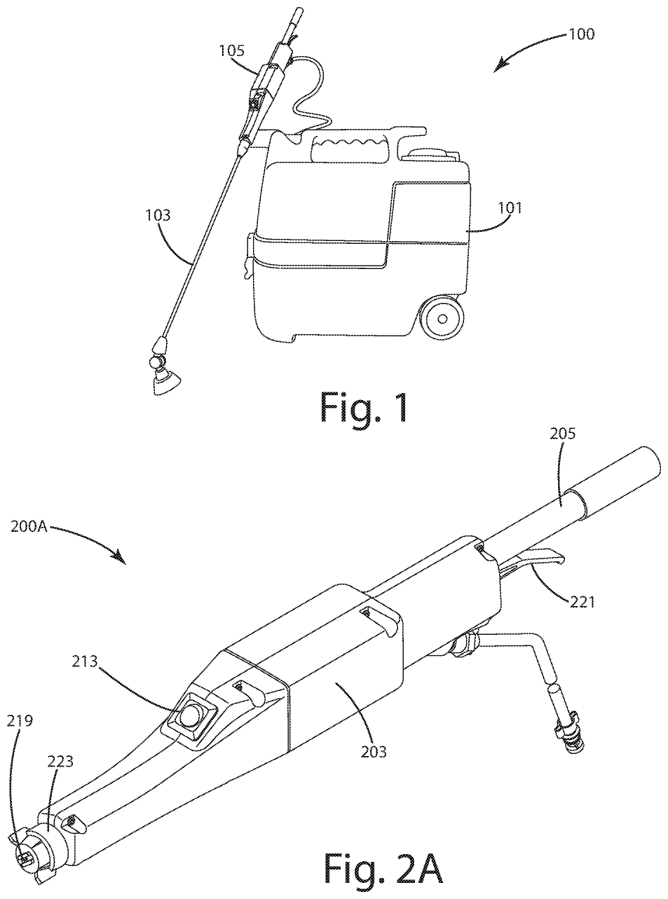

FIG. 1 is a perspective view showing a spraying device with spraying wand. The spraying device 100 includes a sprayer 101 that works to pump cleaning fluid to a electrostatic spraying wand 103. A benefit of the present invention is the electro static spraying wand 103 can be incorporated or retro-fit as an add-on feature to a preexisting disinfectant sprayer. The wand can be used with both mechanical type pump sprayers or more complete motor driven model that use a pump to empty on on-board cleaning fluid reservoir. In other words, an attribute of the present invention is that is can turn any plain liquid sprayer, with little to no changes, into an electrostatic sprayer though the additional of the electrostatic spraying wand as described herein.

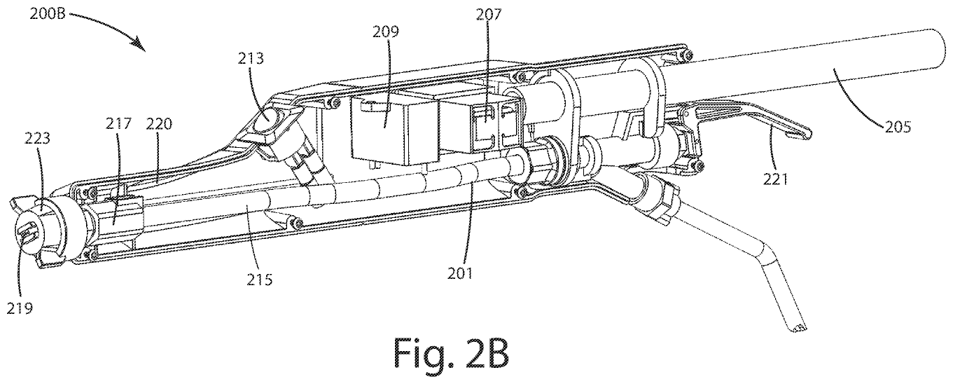

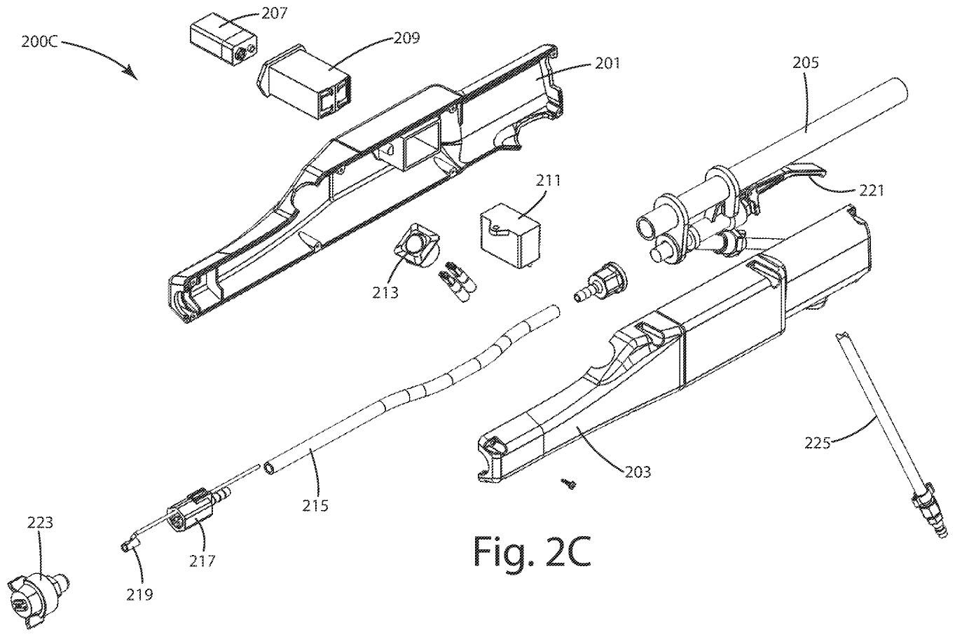

FIG. 2A is an electrostatic spraying wand in accordance with an embodiment of the invention while FIG. 2B is a cross-sectional view of the electrostatic spraying wand as shown in FIG. 2A and FIG. 2C is an exploded view of the electrostatic spraying wand in accordance with FIG. 2A. With regard to FIG. 2A, FIG. 2B and FIG. 2C, the electrostatic spraying wand 200A, 200B, 200C includes and right body 201 and a left body 203 connected by a handle 205. In use, a power source such as an internal battery 207 is configured within a battery holder 209, and operates to power an electro-static generator 211. Power to the electro-static generator 211 is controlled though a switch 213. Those skilled in the art will recognize that an electrostatic generator can be any of various devices used to build up an electric charge to an extreme potential in order to generate electricity,

As water and/or disinfecting fluid moves though a supply line such as solution line 215, it is fed to an electrode housing 217 that houses an electrode 219 therein. The voltage from the electrostatic generator 211 is supplied to the electrode 219 though a wire 220. As the fluid passes though electrode housing 217, it is positively charged by the electrode 219. Although the solution line 215 uses non-conductive tubing, since the fluid is conductive, the cleaning fluid and/disinfecting solution will be charged along it entire supply path, back to the fluid holding tank or reservoir within the sprayer. The electrode may be made of copper or other conductive materials. The pressurized cleaning fluid, from the sprayer, moves though the wand 200A, 200B, 200C through the use of a trigger 221 which controls the fluid through the wand to a nozzle assembly 223. A hose extender 225 is used to bring the fluid to the precise location desired by the user.

Unlike the spray wands used in the prior art, the present invention does not combine a cleaning solution with air which is then atomized by the electrode. Instead, the present invention charges a cleaning solution/water with the electrode with high voltage before it reaches the spray tip. Once the solution passes thru the spray tip, it is converted to a “statically charged” mist. The mist is in the form of tiny droplets that are light weight and able to be attracted to various objects and multi-shaped surfaces. Hence, those skilled in the art will recognize that because the electrostatic generator is located in the wand rather than in the sprayer, or sprayer reservoir, the electrostatic spraying wand 200A, 200B, 200C can operate with many pre-existing sprayer models e.g. electrically powered or pump sprayers that originally did not or do not have an electrostatic spraying capability. Moreover, the electrostatic spraying wand 200A, 200B, 200C is portable and can be used to kill bacteria, pathogens and other disease though the spraying and application of disinfecting solution to objects that might be harbor viruses and bacteria and/or or touched by humans.

In the foregoing specification, specific embodiments of the present invention have been described. However, one of ordinary skill in the art appreciates that various modifications and changes can be made without departing from the scope of the present invention as set forth in the claims below. Accordingly, the specification and figures are to be regarded in an illustrative rather than a restrictive sense, and all such modifications are intended to be included within the scope of present invention. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

We claim:

1. An electrostatic spraying wand comprising:
a housing;
a power source;
an electrostatic generator;
an electrode;
a solution line;
a nozzle assembly; and
wherein the spraying wand can be retro-fit to a preexisting liquid fluid sprayer for allowing dispensed fluid to be electrostatically charged.

2. An electrostatic spraying wand as in claim 1, wherein the power source is a battery stored within the housing.

3. An electrostatic spraying wand as in claim 1, wherein the solution line uses non-conductive tubing.

4. An electrostatic spraying wand as in claim 1, wherein the flow of fluid though the wand is controlled using a trigger.

5. An electrostatic spraying wand as in claim 1, wherein the disinfecting fluid is supplied to the wand from an external spraying device holding the disinfecting fluid.

6. An electrostatic spraying wand as in claim 1, wherein the pre-existing electrostatic sprayer is either a powered or pump sprayer.

7. An electrostatic spraying wand comprising:
a housing;
a power source;
an electrostatic generator;
an electrode;
a solution supply line;
a nozzle assembly; and
wherein the power source powers the electrostatic generator that supplies and substantially high voltage to an electrode that works to charge a disinfecting fluid moving though the electrode, where a charged solution is sprayed though the nozzle assembly to kill pathogens and other germs.

8. An electrostatic spraying wand as in claim 7, wherein the power source is a battery.

9. An electrostatic spraying wand as in claim 7, wherein the power electrode utilizes non-conductive tubing to supply fluid to the nozzle assembly.

10. An electrostatic spraying wand as in claim 7, wherein the flow of fluid though the wand is though using a trigger.

11. An electrostatic spraying wand as in claim 7, wherein the disinfecting fluid is supplied to the wand from a spraying device holding the disinfecting fluid.

12. An electrostatic spraying wand as in claim 11, wherein the spraying device is a powered or pump sprayer.

13. An electrostatic spraying wand comprising:
an elongated housing;
a battery powered source within the housing;
an electrostatic generator;
an electrode connected to the electrostatic generator;
a solution supply line;
a nozzle assembly connected to the non-conductive solution line; and
wherein the power source powers the electrostatic generator for supplying a substantially high voltage to an electrode that works to charge a disinfecting fluid moving though the electrode, where a charged solution is sprayed though the nozzle assembly to kill pathogens and other germs.

14. An electrostatic spraying wand as in claim 13, wherein the power electrode uses non-conductive tubing for supplying fluid to the nozzle assembly.

15. An electrostatic spraying wand as in claim 13, wherein the flow of fluid though the wand is controlled using a trigger attached to the housing.

16. An electrostatic spraying wand as in claim 13, wherein the disinfecting fluid is supplied to the wand from an attached spraying device holding the disinfecting fluid.

17. An electrostatic spraying wand as in claim 16, wherein the spraying device is a powered or pump sprayer.
